(19) United States
(12) Patent Application Publication
(10) Pub. No.: US 2022/0285012 A1
(43) Pub. Date: Sep. 8, 2022

(54) METHOD AND SYSTEM FOR ENFORCING FACILITY OCCUPANCY

(71) Applicant: Xerox Corporation, Norwalk, CT (US)

(72) Inventors: Fritz Francis Ebner, Pittsford, NY (US); Gareth Ratcliffe, Denver, CO (US); Matthew Dylan Coene, Ontario, NY (US); Raviteja Gunda, Webster, NY (US)

(21) Appl. No.: 17/189,361

(22) Filed: Mar. 2, 2021

(51) Int. Cl. G16H 40/20; G16H 50/30; G06Q 10/06; G06Q 50/16; G06Q 50/26; G07C 9/28; G07C 9/27; G16H 50/70; G16H 50/80; H04W 4/029; H04W 4/021; G08B 5/22; G08B 21/02; H04W 4/33

(52) U.S. Cl. CPC G16H 40/20; G16H 50/30; G06Q 10/0635; G06Q 50/163; G06Q 50/265; G07C 9/28; G07C 9/27; G16H 50/70; G16H 50/80; G06Q 10/063118; H04W 4/029; H04W 4/021; G08B 5/22; G08B 21/02; H04W 4/33

(57) ABSTRACT

A system for enforcing occupancy for a facility includes client electronic devices, each associated with one or more individuals who have access to a facility, an access monitoring system, and a computing device in communication with at least a portion of the plurality of the client electronic devices and the access monitoring system. The system determines, for the facility, a health risk rating, a maximum occupancy, a risk-adjusted occupancy, an estimated occupant number, and determines, based on the risk-adjusted occupancy and the estimated occupant number whether one or more individuals need to exit the facility. If individuals need to exit the facility, the system identifies and notifies the individuals.

### BACKGROUND

Controlling facility occupancy in times of emergency or other critical periods (e.g., during disease spread) is a central concern for facility managers and company health personnel. Often, a decision as to whether to allow an individual to enter a facility is based on access credentials and capacity constraints. However, in certain situations, information pertaining to population risk, should also be considered in managing facility occupancy.

This document describes methods and systems that are directed to addressing the issues listed above.

### SUMMARY

In an embodiment, a system for enforcing occupancy for a facility includes a plurality of client electronic devices, each associated with one or more individuals who have access to a facility, an access monitoring system, and a computing device in communication with at least a portion of the plurality of the client electronic devices and the access monitoring system. The system includes a computer-readable storage medium having one or more programming instructions that, when executed, cause the computing device to determine a health risk rating for a facility, determine a maximum occupancy for the facility, determine a risk-adjusted occupancy for the facility by adjusting the maximum occupancy based on the health risk rating, determine an estimated occupant number for the facility during a time period using the access monitoring system, determine, based on the risk-adjusted occupancy and the estimated occupant number, whether one or more individuals need to exit the facility, in response to determining that one or more individuals need to the exit the facility, identify the one or more individuals from a plurality of individuals who are present at the facility at the time period, and notify the one or more individuals who are identified to exit the facility.

The access monitoring system may include an indoor location tracking system having one or more wireless access points in communication with one or more of the plurality of client electronic devices.

The access monitoring system may include a geofence system in communication with at least a portion of the plurality of client electronic devices. The geofence system may include one or more receivers configured to receive location-based data from one or more of the plurality of client electronic devices.

The access monitoring system may include a physical access control system having one or more reader devices that are configured to receive one or more access credentials from one or more individuals seeking access to the facility.

The system may determine a health risk rating for a facility by accessing information from one or more health metrics data stores associated with the facility, and applying a framework to at least a portion of the information that is accessed. The information from the one or more health metrics data stores may include a trend value associated with a health condition over a period of time and for a geographic location, a measure of a number of cases of the health condition over the period of time and for the geographic location, and a positivity rate associated with the health condition over the period of time and for the geographic location.

The system may determine a maximum occupancy for the facility by identifying the maximum occupancy for the facility from one or more capacity data stores associated with the facility.

The system may determine a risk-adjusted occupancy for the facility by determining whether the health risk rating for the facility is at a highest level, in response to the health risk rating for the facility being at the highest level, identifying an essential personnel capacity for the facility, and assigning the essential personnel capacity to the risk-adjusted occupancy. The essential personnel capacity may represent a number of essential works for the facility.

The system may determine a risk-adjusted occupancy for the facility by determining whether the health risk rating for the facility is at a lowest level, in response to the health risk rating for the facility being at the lowest level, identifying a normal capacity for the facility, and assigning the normal capacity to the risk-adjusted occupancy.

The system may determine a risk-adjusted occupancy for the facility by determining whether the health risk rating for the facility is between a lowest level and a highest level, in response to the health risk rating for the facility being between a lowest level and a highest level, identifying a normal capacity for the facility, and assigning a certain percentage of the normal capacity to the risk-adjusted occupancy.

The system may determine an estimated occupant number for the facility at the time period by (a) identifying a number of individuals who have submitted access requests to access the facility during the time period; (b) receiving, from an indoor location tracking system, an estimate of a number of electronic devices in communication with one or more wireless access points at the facility during the time period; (c) receiving, from a geofence system, an estimate of a number of electronic devices within a geofence during that time period, wherein the geofence encompasses the facility; or (d) receiving an estimate of a number of individuals who have presented one or more access credentials to one or more reader devices at the facility over the time period.

The system may determine an estimated occupant number for the facility at a time period by identify a first estimate as a number of individuals who have submitted access requests to access the facility during the time period, receiving, from an indoor location tracking system, a second estimate comprising an estimate of a number of electronic devices in communication with one or more wireless access points at the facility during the time period, receiving, from a geofence system, a third estimate comprising a number of electronic devices within a geofence during that time period, wherein the geofence encompasses the facility, receiving a fourth estimate of a number of individuals who have presented one or more access credentials to one or more reader devices at the facility over the time period, and averaging the first estimate, the second estimate, the third estimate, and the fourth estimate.

The system may determine, based on the risk-adjusted occupancy and the estimated occupant number, whether one or more individuals need to exit the facility by determining whether the risk-adjusted occupancy is greater than the estimated occupant number.

The system may identify the one or more individuals from a plurality of individuals who are present at the facility at the time period by identifying the one or more individuals as those associated with a priority designation for the facility that indicates they are not essential personnel.

The system may notify the one or more individuals who are identified to exit the facility by causing an email to be sent to an email address of each of the one or more individuals who are identified, causing a text message to be sent to an electronic device of each of the one or more individuals who are identified, or causing a push notification to be displayed on an electronic device of each of the one or more individuals who are identified.

### BRIEF DESCRIPTION OF THE DRAWINGS

FIG. 1 illustrates an example system for enforcing facility occupancy.

FIG. 2 illustrates a flow chart of an example method of managing occupancy.

FIG. 3 illustrates an example framework.

FIG. 4 illustrates an example indoor location tracking system.

FIG. 5 illustrates an example physical access control system.

FIG. 6 illustrates an example access request.

FIG. 7 illustrates a block diagram of example hardware that may be used to contain or implement program instructions.

### DETAILED DESCRIPTION

As used in this document, the singular forms “a,” “an,” and “the” include plural references unless the context clearly dictates otherwise. Unless defined otherwise, all technical and scientific terms used in this document have the same meanings as commonly understood by one of ordinary skill in the art. As used in this document, the term “comprising” (or “comprises”) means “including (or includes), but not limited to.” When used in this document, the term “exemplary” is intended to mean “by way of example” and is not intended to indicate that a particular exemplary item is preferred or required.

In this document, when terms such “first” and “second” are used to modify a noun, such use is simply intended to distinguish one item from another, and is not intended to require a sequential order unless specifically stated. The term “approximately,” when used in connection with a numeric value, is intended to include values that are close to, but not exactly, the number. For example, in some embodiments, the term “approximately” may include values that are within +/−10 percent of the value.

Additional terms that are relevant to this disclosure will be defined at the end of this Detailed Description section.

This disclosure generally relates to a system that combines relevant disease statistics, facility capacity, and real-time facility usage metrics to control the overall facility occupancy.

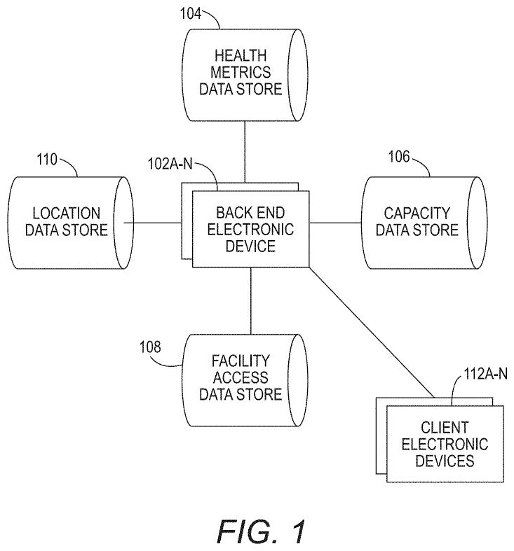

FIG. 1 illustrates an example system for enforcing facility occupancy according to an embodiment. As illustrated in FIG. 1, a system 100 may include one or more backend computing devices 102a-N. In situations where more than one backend computing device is utilized, the backend computing devices may be located at the same physical location (e.g., a server room). In alternate embodiments, one or more of the backend computing devices may be located remotely from one or more other backend computing devices (e.g., distributed servers).

In various embodiments, the backend computing devices may be associated with an owner, operator, user, and/or the like of a facility. For example, a business may occupy a building and the company may own or have the right to use the backend computing devices.

As illustrated in FIG. 1, one or more of the backend computing devices 102a-N may include or be in communication with one or more data stores that may store data or information for assessing occupancy of a facility. For example, one or more backend computing devices 102a-N may be in communication with one or more health metrics data stores 104a-N. A health metrics data store may be maintained by an applicable third party, such as, for example, a health department, a county and/or the like. A health metrics data store 104a-N may store information pertaining to one or more diseases, viruses, health conditions and/or the like. Such diseases, viruses, health conditions and/or the like may be contagious conditions that may be spread from one individual to another. The stored information may be particular to a certain region such as, for example, a borough or municipality, a township, a county, a city or town, a state, a country, and/or the like.

In various embodiments, a health metrics data store 104a-N may store information pertinent to a geographical region in which a facility is located. For example, if a facility of interest is located in Chicago, one or more backend computing devices may be in communication with one or more health metrics data stores associated with Cook County.

The information stored by a health metrics data store 104a-N may include metrics associated with a particular disease or health condition in the applicable geographic location. For instance, a health metrics data store 104a-N may store information about how many individuals in the applicable geographic location have been diagnosed with a health condition over a certain time period, a number of individuals who have died due to the health condition over a certain time period, a number of individuals who have been tested for the health condition over a certain time period, a positivity rate of the health condition over a certain time period, a trend associated with the health condition (e.g., whether the number of health conditions in the applicable area is trending up, down, or staying flat), an indication of whether a number of cases of the health condition exceeds a threshold value, and/or the like.

Referring back to FIG. 1, the system 100 may include one or more capacity data stores 106a-N. A capacity data store 106a-N may store capacity or occupancy information associated with one or more facilities or portions of facilities. The occupancy information may include a maximum number of occupants of a facility (or portion of a facility). The maximum number of occupants of a facility is referred to in this disclosure as a “normal capacity.” The occupancy information may include an indication of a number of essential workers for a facility. Essential workers are those individuals who are permitted to occupy a facility despite the existence of an emergency or health-related situation. The determination of who qualifies as an essential worker may be made by an employer, the government, or a combination of the two. As an example, an essential worker may be an individual that performs work involving the safety of human life, the protection or property, or whose tasks are essential to critical infrastructure operations.

A capacity data store 106a-N may store one or more priority designations associated with one or more individuals who have access rights to one or more facilities. Examples of priority designations may include, without limitation, “essential” and “non-essential”. Additional and/or alternate designations may be used within the scope of this disclosure.

In various embodiments, the occupancy information may be time-specific. For example, a maximum occupancy of a facility may have a certain value at a certain time, and a different value at a different time. A capacity data store 106a-N may store a maximum occupancy and a corresponding time period.

In various embodiments, one or more backend computing devices 102a-N may include one or more capacity data stores 106a-N. In other embodiments, one or more backend computing devices 102a-N may be in communication with one or more capacity data stores 106a-N.

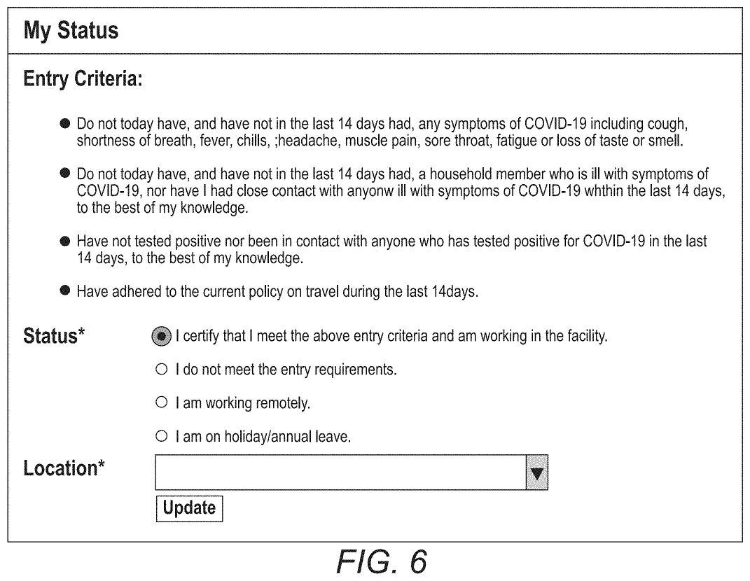

As illustrated in FIG. 1, the system 100 may include one or more facility access data stores 108a-N. A facility access data store 108a-N may store information about individuals who are requesting access to a facility and/or individuals who have visited a facility over a certain time period. For example, an employee wishing to access a facility may submit an access request. The access request may be submitting electronically via an electronic device of the employee such as, for example, a mobile phone, a tablet, a laptop, or other computing device. The access request may be sent to one or more backend computing devices 102a-N. Information pertaining to the access request such as, for example, the employee's name, title, facility, time, date, and/or other information may be stored in a facility access data store 108a-N. If multiple facilities are at issue, each facility may be associated with a separate facility access data store 108a-N. In other embodiments, one or more facility access data stores 108a-N may be used that are indexed based on the corresponding facility. FIG. 6 illustrates an example access request according to an embodiment.

In various embodiments, a facility access data store 108a-N may store information about individuals who do access a particular facility. As described in more detail below, a facility may have an access control system. For example, to gain physical access to a facility, an individual may be required to present a token such as, for instance, credentials, a fob, a badge or access card, and/or the like. The token may store information specific to the individual, such as, for example, the individual's name, title, and/or the like. An individual may present a token to an electronic scanning device or reader, which may read information from the token. The information that is read from the token as well as other relevant information (e.g., an identifier associated with the reader that read the token, a timestamp, and/or the like), may be stored by one or more facility access data stores 108a-N.

As illustrated in FIG. 1, the system 100 may include a location data store 110a-N. A location data store 110a-N may store information about a location of one or more individuals within a facility. As described in more detail below, a location of an individual within a facility may be provided by the individual, geofenced with a mobile electronic device application, via an indoor location system and/or the like. A location data store 110a-N may store an indicator of an individual's location within a facility along with a timestamp associated with that location.

In various embodiments, as described throughout this disclosure, one or more backend computing devices 102a-N may be in communication with one or more client electronic devices 112a-N, such as, for example, one or more mobile electronic devices.

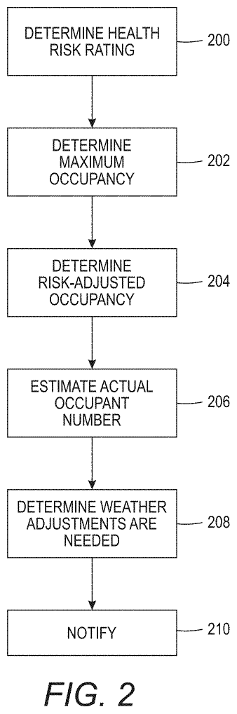

FIG. 2 illustrates a flow chart of an example method of enforcing occupancy for a facility according to an embodiment. As illustrated by FIG. 2, the system may determine 200 a health risk rating for one or more facilities. A health risk refers to an indicator of risk associated with being exposed to a particular health condition and/or contracting a particular health condition in the case of contagious health conditions.

The system may determine 200 a health risk rating for a facility by accessing information from one or more health metrics data stores associated with the facility and applying a framework to at least a portion of the accessed information to determine a health risk rating.

For example, a health risk rating may be determined based on a composite of a trend value associated with the health condition over a period of time and for a certain geographic location, a measure of a number of cases of the health condition in an applicable time period and geographic location, and a positivity rate associated with the health condition over a period of time and for a certain geographic location. The system may access this information from one or more applicable health metric data stores, and may apply a framework to the data to determine a health risk rating. The framework may specify a health risk rating for a particular combination of data values.

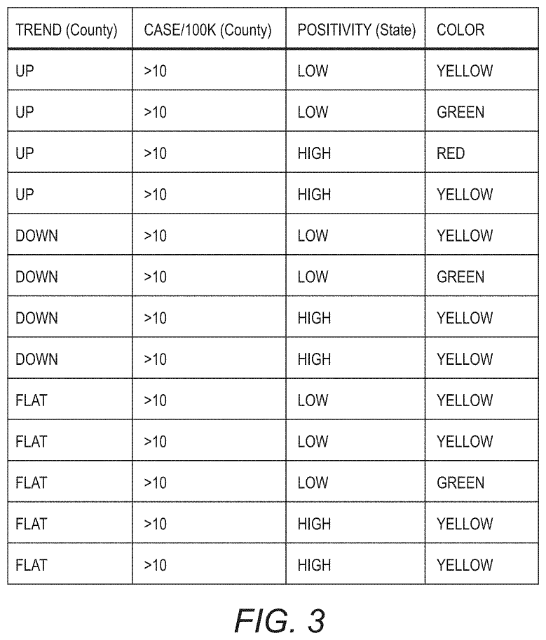

FIG. 3 illustrates an example framework according to an embodiment. As illustrated in FIG. 3, the framework considers the trend of a health condition (up, down, or flat), an indication of whether the number of cases of the health condition per 100,000 people in the applicable county is greater than or less than a threshold value (in this example, the threshold value is 10), and indication of a positivity rate associated with the health condition in the state (low or high). It is understood that additional and/or alternate frameworks or framework criteria may be used within the scope of this disclosure.

For example, as shown by FIG. 3, the framework indicates that a trend of “up”, an indication that the number of cases is greater than the threshold value, and a positivity rate of “low” yields a health risk rating of “yellow”. In comparison, a trend of “up”, an indication that the number of cases is greater than the threshold value, and a positivity rate of “high” yields a health risk rating of “red.”

With respect to the framework illustrated by FIG. 3, the health risk ratings are “green”, “yellow”, and “red”, where a rating of “green” indicates a risk less severe than that of “yellow” or “red”, a rating of “yellow” indicates a risk with a severity between that of “green” and red, and “red” indicates a risk higher than that of “green” and “yellow”. Additional and/or alternate risk ratings and/or risk rating values may be used within the scope of this disclosure.

In various embodiments, a framework may be stored in one or more data stores. The system may access the framework and apply it to data that is accessed from one or more health metrics data stores to determine a health risk rating for a facility. The information that the system may access from one or more health metrics data stores may be data associated with the geographical location of the applicable facility (e.g., the county, town, city, township, state, and/or other geographical location where the facility is located).

Referring back to FIG. 2, the system may determine 202 a maximum occupancy for the facility. The system may determine 202 a maximum occupancy for a facility by accessing this information from one or more capacity data stores. For instance, the system may search one or more capacity data stores for the particular facility and may identity the maximum occupancy for the facility from the capacity data stores.

The system may determine 204 risk-adjusted occupancy for the facility. The risk adjusted occupancy may be an occupancy limit for a facility that represents the maximum occupancy for the facility adjusted based, at least in part, on the applicable health risk rating for the facility.

A risk-adjusted occupancy for a facility may be set to accommodate essential personnel only if the health risk rating for the facility is at a highest level. For example, if the system determines that the health risk rating for a facility is at its highest level, the system may retrieve the essential personnel capacity for the facility from one or more capacity data stores, and may set the risk-adjusted occupancy for the facility to the essential personnel capacity.

In various embodiments, if a health risk rating for a facility is at its lowest level, the system may set a risk-adjusted occupancy for the facility to the maximum number of occupants for the facility. For example, the system may retrieve the normal capacity for the facility from one or more capacity data stores, and may set the risk-adjusted occupancy for the facility to the normal capacity.

If a health risk rating for a facility is between its lowest level and its highest level, the system may set a risk-adjusted occupancy for the facility to a percentage of the normal capacity for the facility. Referring to above example, a health risk rating may be “yellow”, which may be a rating between “red” (more severe) and “green” (less severe). The system may set a risk-adjusted occupancy for the facility to 50% of the normal capacity in this situation. Other percentages may be used within the scope of this disclosure.

In various embodiments, the adjustment percentage may be stored in one or more data stores in association with the health risk rating to which it corresponds. Table 1 below illustrates example adjustment percentages associated with example health risk ratings. However, it is understood that additional and/or alternate health risk ratings and/or adjustment percentages may be used within the scope of this disclosure.

TABLE 1

| Health risk rating | Adjustment percentage |
|---|---|
| Red | 0% of normal capacity |
| Orange | 25% of normal capacity |
| Yellow | 50% of normal capacity |
| Blue | 75% of normal capacity |
| Green | 100% of normal capacity |

Referring back to FIG. 2, the system may estimate 206 an actual occupant number for a facility. The actual occupant number refers to a number of individuals present at or in a facility at a particular time or time period. In various embodiments, the system may estimate 206 an actual occupant number for a facility using one or more access monitoring systems for the facility.

An access monitoring system may include one or more data stores that may store information about access requests and/or attempts. The system may estimate 206 an actual occupant number for a facility by retrieving information from one or more facility access data stores for a particular time or time period. As explained above with respect to FIG. 1, a facility access data store may store information about individuals who are requesting access to a facility over a certain time period.

For example, an employee wishing to access a facility may submit an access request. The access request may be submitting electronically via an electronic device of the employee such as, for example, a mobile phone, a tablet, a laptop, or other computing device. The access request may be sent to one or more backend computing devices. Information pertaining to the access request such as, for example, the employee's name, title, facility, time, date, and/or other information may be stored in a facility access data store. Table 2 illustrates example information that may be store in a facility access data store according to an embodiment.

TABLE 2

| | | Requested | Requested |
|---|---|---|---|
| Individual | Facility | access date | access time |
| User A | Facility X | January 20 | 8am-noon |
| User B | Facility X | January 20 | 9am-noon |
| User C | Facility X | January 20 | 1pm-5pm |
| User D | Facility X | January 20 | 8am-5pm |

The system may use the number of individuals who have requested access to a facility as an estimate of the actual occupant number for a facility for a time period. For example, a system may estimate based on the information in Table 2 that the actual occupant number for Facility X between 8 am and 5 pm on January 20 is four individuals.

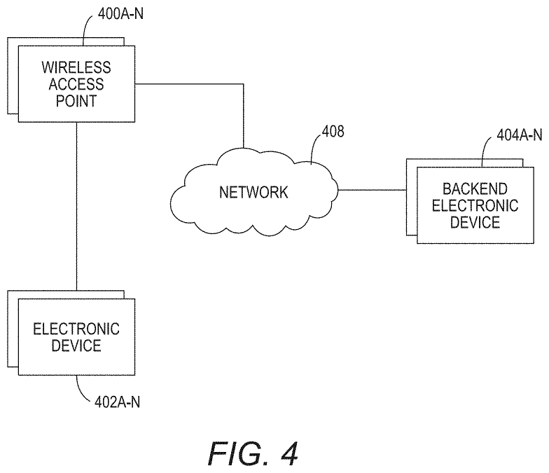

In various embodiments, an access monitoring system may include a wireless-based indoor location tracking system that may be used to estimate an actual occupant number for a facility. FIG. 4 illustrates an example indoor location tracking system according to an embodiment. As illustrated by FIG. 4, an indoor location tracking system may include one or more wireless access points 400. A wireless access point 400 refers to a hardware electronic device that permits a wireless-enabled electronic device to connect to a wired network. A wireless access point 400 may be a standalone device which is positioned at various locations in an indoor environment such as, for example, a facility. Alternatively, a wireless access point 400 may be a component of another device, such as, for example, a router which is similarly positioned throughout an environment. The wireless access points 400 may be present in a high enough density to service an entire environment.

In various embodiments, a wireless access point 400a-N may log the time and the strength of one or more communications from one or more wireless-enabled electronic devices 402a-N in an indoor environment. The wireless access point 400a-N may send at least part of the logged information to an electronic device such as, for example, a backend electronic device 404a-N, via one or more networks 408. The backend electronic device 404a-N may use the received information to estimate a location of an electronic device 402. For example, a backend electronic device 404a-N may use the received information to determine a position of an electronic device 402 relative to a fixed point in the environment. A backend electronic device may store or have access to a map of a relevant environment, and may use the map to determine a position of a wireless-enable electronic device relative to a reference point. This position may be measured as a certain distance from a reference point, or as one or more position coordinates, such as longitude and latitude.

In various embodiments, an indoor location tracking system, such as the one described with respect to FIG. 4, may use low accuracy and high latency WiFi location tracking techniques to establish an initial position of a mobile electronic device in an indoor environment.

The system may utilize an indoor location tracking system, such as the one described with respect to FIG. 4, to estimate the number of wireless-enabled electronic devices in a facility at a certain time or over a time period. For instance, the system may determine a number of wireless-enabled electronic devices in a facility that are in communication with one or more wireless access points in the facility at a certain time or over a time period. The system may consider this number to represent an actual occupant number for the facility.

In various embodiments, access monitoring system may include a geofencing system that may be used to estimate a number of actual occupant number for a facility. A geofencing system refers to a system that provides a location-based service in which software uses location-based data (e.g., global positioning system (GPS) data, radio frequency identification (RFID) data, wireless (Wi-Fi) data, and/or cellular data) to trigger an action when an enable electronic device enters or exits a geofence. A geofence refers to a virtual boundary that is established around a geographical location.

For example, a geofence may be established around a facility. The geofence system may include one or more receivers that receive location-based data from one or more of the plurality of client electronic devices. When the geofence system detects that a mobile electronic device has entered the geofence, the geofencing system may increase an estimated number of individuals within the facility by one. Similarly, when the geofence system detects that a mobile electronic device has left the geofence, the geofencing system may reduce an estimated number of individuals within the facility by one. The system may use the geofence system's estimated number of individuals within a facility to represent the actual occupant number for the facility.

In various embodiments, an access monitoring system may include a physical access control system that is used to estimate an actual occupant number for a facility at a certain time or over a time period. A physical access control system refers to a system that controls individual access to a facility via electronic authentication of one or more credentials.

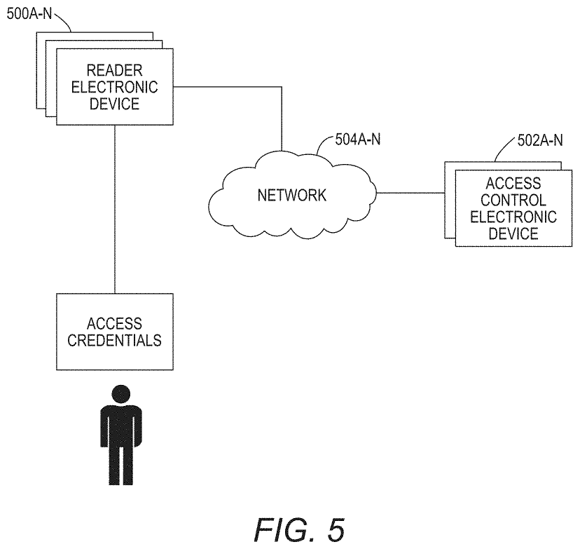

FIG. 5 illustrates an example physical access control system according to an embodiment. As illustrated by FIG. 5, the system may include one or more reader devices 500a-N, which may be positioned at one or more access points of a facility. A reader device refers to a physical device that is configured to receive one or more access credentials from an individual.

A reader device may include one or more optical readers, which may be a device that captures visual information and translates the image into digital information. For instance, an individual may present an access token that contains access credential information. The reader device may capture this information from the access token. A token may include a badge, an access card, and/or the like.

For example, a reader device may include a barcode reader that may scan a barcode on a token. As another example, a reader device may include a Quick Response (QR) reader, which may scan a QR code on a token.

In various embodiments, a reader device may include one or more biometric readers. A user may present a biometric to the reader device such as, for example, a fingerprint scan, an eye scan, a face scan, and/or the like.

A reader device may include an input device such as, for example, a keypad or touchscreen. A user may provide one or more unique access credentials to the reader device via the input device. For instance, a user may type a password or personal identification number using a keypad or touchscreen.

A reader device may include a short-range transmitter, receiver and/or transceiver that may send and/or receive access credential information from a token when the token is in range. A receiver, a transmitter, and/or a transceiver may be a near-field communication (NFC) or other short-range communication receiver, transmitter, and/or transceiver such as, for example, a radio frequency identification (RFID) coil, an NFC antenna, and/or the like. In various embodiments, a receiver, transmitter, and/or transceiver may be part of an integrated circuit such as, for example, an RFID chip, and NFC chip, and/or the like.

It is understood that additional and/or alternate reader devices and/or access credentials may be used within the scope of this disclosure.

Referring back to FIG. 5, a physical access control system may include one or more server computing devices 502a-N. One or more access control electronic devices 502a-N may be in communication with one or more reader devices 500a-N via one or more communication networks 504a-N. In various embodiments, a reader device 500a-N may transmit at least a portion of the access credential information that is received by it to one or more access control electronic devices 502a-N. The one or more access control electronic devices 502a-N may store at least a portion of the access credential information in one or more data stores. This access credential information may be stored in association with a timestamp indicating when the access credential information was presented. The system may use the volume of access credential information that is stored as an indication of a number of individuals in a facility over a time period.

For example, an individual may present access credential information to a reader device to gain access to a facility, and may present access credential information to a reader device to exit a facility. The system may compare a current date and time against the timestamps associated with access credential information to estimate a number of people in a facility as of the current date and time.

As another example, the system may estimate a number of individuals present at a facility based on access credential information that is presented to gain access to the facility. In other words, an individual may not need to provide access credentials information to exit a facility. In this situation, the system may assume that an individual spends a particular amount of time or time period at the facility before exiting and may estimate occupancy based on this number. For instance, a system may assume that an individual spends on average four hours at a facility and may estimate that an individual is on site at a facility for a period of four hours after presenting access credential information. Additional and/or alternate periods of time may be used within the scope of this disclosure.

In various embodiment, the system may estimate an actual occupant number for a facility using multiple methods, and may determine estimate the actual occupant number by averaging the results of the different methods. For example, the system may estimate an actual occupant number by averaging the results received from a facility using a wireless-based indoor location tracking system, a geofence system, and a physical access control system. For instance, using the example results illustrated in Table 3, the system may estimate the actual occupant number for a facility as 81 (i.e., (72+79+101+72)/4).

TABLE 3

| System | Estimated occupant number |
|---|---|
| Access requests | 72 |
| Indoor location tracking | 79 |
| system | |
| Geofence system | 101 |
| Physical access control system | 72 |

In various embodiments, the system may estimate an actual occupant number for a facility based on a weighted average of the estimates provided by various sources. For instance, for a particular facility, the estimate provided by an indoor location tracking system may be more reliable than a geofence system and a physical access control system, and its results may be weighted to account for this. Similarly, the results provide by a physical access control system may be more reliable than an estimate from a geofence system, and its results may be weighted to account for this. Other weight and reliabilities may be used within the scope of this disclosure.

Table 4 illustrates an example situation illustrating associated weight values associated with the source of estimates of actual occupant numbers for a facility. The system may estimate the actual occupant number for the facility of Table 4 as 80.6 (i.e., (0.20 (72)+0.40 (79)+0.20 (101)+0.20 (72))/4). Additional and/or alternate weights and/or sources may be used within the scope of this disclosure.

TABLE 4

| System | Weight | Estimated occupant number |
|---|---|---|
| Indoor location | 0.50 | 79 |
| tracking system | | |
| Geofence system | 0.20 | 101 |
| Physical access | 0.30 | 72 |
| control system | | |

The value of the weights that are assigned to each source of occupancy estimates may be determined by those running or operating a facility. The applicable weights may be stored in one or more data stores that are accessible by the system.

Referring back to FIG. 2, the system may determine 208 whether any adjustments to the estimated actual occupant number for a facility should be made. An adjustment may include, without limitation, allowing one or more individuals to continue to submit access requests to enter a facility, allowing one or more individuals to enter a facility, prohibiting one or more individuals from submitting access requests to enter a facility, prohibiting one or more individuals access to enter a facility, notifying one or more individuals that they need to exit a facility, and/or the like.

The system may determine 208 whether an adjustment to an estimated actual occupant number for a facility should be made by comparing the risk adjusted occupancy for the facility to the estimated actual occupant number for the facility.

If the risk adjusted occupancy for a facility is greater than the estimated actual occupant number for the facility, then the system may permit one or more individuals to submit access requests to enter the facility and/or physically enter the facility.

If the risk adjusted occupancy for a facility is less than or equal to the estimated actual occupant number for the facility, the system may deny an access request from one or more individuals for a facility unless the individual is associated with a certain priority designation. For instance, when a user who is associated with a designation of “non-essential” attempts to complete or submit an access request for a facility, the system may notify the user that he or she is not permitted to submit the request and should not attempt to gain physical access to the facility. However, a user who is associated with a designation of “essential” may be permitted to submit an access request to enter a facility.

As described above, when a user completes an access request, the user may provide an identifier such as, for example, his or her name, employee number, or other identifier. The system may use this identifier to search a data store, such as, for example, the capacity data store as described above with respect to FIG. 1, to determine a priority designation associated with the individual. If the user's priority designation does not match one or more permitted designations for the facility, the user's access request may be denied. If the user's priority designation does match one or more permitted designations for the facility, the user's access request may be granted.

If the risk adjusted occupancy for a facility is less than or equal to the estimated actual occupant number for the facility, the system may not allow one or more individuals to access or enter a facility unless they are associated with a certain priority designation. For instance, if an individual attempts to access facility by providing access credential information to a reader device, for example, the system may deny entry to the individual and may not unlock an entry door or other access point in response to the user providing his or her access credential information unless the individual is associated with one or more permitted designations for the facility.

As another example, if the system detects that an individual is within proximity of a facility (e.g., within a geofence of a facility), the system may cause a notification to be displayed on the individual's electronic device (e.g., a mobile phone), alerting the individual that he or she is not permitted access to the facility unless the individual is associated with one or more permitted designations for the facility.

In various embodiments, if the risk adjusted occupancy for a facility is less than or equal to the estimated actual occupant number for the facility, the system may notify 210 a certain number of individuals who are currently at the facility that they must leave the facility. The system may determine a number of individuals to notify by subtracting the risk adjusted occupancy from the estimated actual occupant number.

The system may determine which individuals to notify by analyzing the priority designations associated with the individuals who are at the facility. For instance, the system may identify a certain number of individuals who are associated with a priority designation other than “essential” and may notify those individuals that they need to vacate the premises of the facility.

The system may notify an individual by sending an email notification to an email address associated with the individual, sending a text message to a phone number of an electronic device associated with the individual, or causing an alert or notification to be displayed on a display of an electronic device associated with the individual (e.g., a push notification) such as, for example, a mobile phone. In various embodiments, one or more modes of notification associated with one or more individuals may be stored in one or more data stores that are accessible by the system. The modes of notification may include, without limitation, an email address, a phone number, a unique identifier associated with an electronic device, and/or the like.

In various embodiments, the alerts may include text, images, graphics, audio sounds, and/or the like. The alerts may instruct the individual to vacate the premises of the facility as soon as possible, within a certain time period, by a certain time, and/or the like.

If the risk adjusted occupancy for a facility is less than or equal to the estimated actual occupant number for the facility, the system may generate one or more notifications to site administrators, managers, monitors, and/or the like notifying them that capacity has been exceeded. The notifications may be text messages, email messages, push notifications and/or the like.

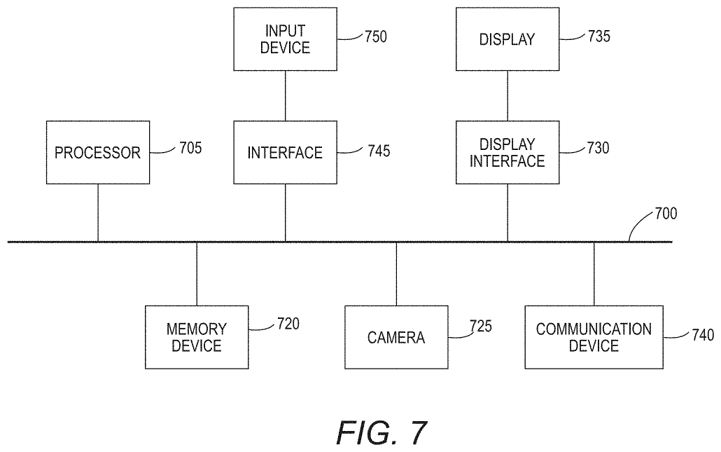

FIG. 7 depicts an example of internal hardware that may be included in one or more electronic devices, such as an electronic device of a system for enforcing occupancy for a facility or an access monitoring system as described above, or one or more other electronic devices. An electrical bus 700 serves as an information highway interconnecting the other illustrated components of the hardware. Processor 705 is a central processing device of the system, configured to perform calculations and logic operations required to execute programming instructions. As used in this document and in the claims, the terms “processor” and “processing device” may refer to a single processor or any number of processors in a set of processors that collectively perform a set of operations, such as a central processing unit (CPU), a graphics processing unit (GPU), a remote server, or a combination of these. The programming instructions may be configured to cause the processor to perform any or all of the methods described above. Read only memory (ROM), random access memory (RAM), flash memory, hard drives and other devices capable of storing electronic data constitute examples of memory devices 720. A memory device may include a single device or a collection of devices across which data and/or instructions are stored.

An optional display interface 730 may permit information from the bus 700 to be displayed on a display device 735 in visual, graphic or alphanumeric format. An audio interface and audio output (such as a speaker) also may be provided. Communication with external devices may occur using various communication devices 740 such as a wireless antenna, an RFID tag and/or short-range or near-field communication transceiver, each of which may optionally communicatively connect with other components of the device via one or more communication system. The communication device 740 may be configured to be communicatively connected to a communications network, such as the Internet, a local area network or a cellular telephone data network.

The hardware may also include a user interface sensor 745 that allows for receipt of data from input devices 750 such as a keyboard, a mouse, a joystick, a touchscreen, a touch pad, a remote control, a pointing device and/or microphone. Such devices may be used to help label images in training the model. Digital image frames also may be received from a camera 725 that can capture video and/or still images.

Terminology that is relevant to this disclosure includes:

An “electronic device” or a “computing device” refers to a device or system that includes a processor and memory. Each device may have its own processor and/or memory, or the processor and/or memory may be shared with other devices as in a virtual machine or container arrangement. The memory will contain or receive programming instructions that, when executed by the processor, cause the electronic device to perform one or more operations according to the programming instructions. Examples of electronic devices include personal computers, servers, mainframes, virtual machines, containers, gaming systems, televisions, digital home assistants and mobile electronic devices such as smartphones, personal digital assistants, cameras, tablet computers, laptop computers, media players and the like. Electronic devices also may include components of vehicles such as dashboard entertainment and navigation systems, as well as on-board vehicle diagnostic and operation systems. In a client-server arrangement, the client device and the server are electronic devices, in which the server contains instructions and/or data that the client device accesses via one or more communications links in one or more communications networks. In a virtual machine arrangement, a server may be an electronic device, and each virtual machine or container also may be considered an electronic device. In the discussion above, a client device, server device, virtual machine or container may be referred to simply as a “device” for brevity. Additional elements that may be included in electronic devices are discussed above in the context of FIG. 7.

A “facility” refers to a physical location or structure having an occupancy load. Example facilities may include, without limitation, buildings, floors or other areas or portions of buildings, warehouses, tents, enclosures, and/or the like.

The terms “processor” and “processing device” refer to a hardware component of an electronic device that is configured to execute programming instructions. Except where specifically stated otherwise, the singular terms “processor” and “processing device” are intended to include both single-processing device embodiments and embodiments in which multiple processing devices together or collectively perform a process.

The terms “memory,” “memory device,” “data store,” “data storage facility”, “computer-readable storage medium” and the like each refer to a non-transitory device on which computer-readable data, programming instructions or both are stored. Except where specifically stated otherwise, the terms “memory,” “memory device,” “data store,” “data storage facility”, “computer-readable storage medium”, and the like are intended to include single device embodiments, embodiments in which multiple memory devices together or collectively store a set of data or instructions, as well as individual sectors within such devices.

In this document, the terms “communication link” and “communication path” mean a wired or wireless path via which a first device sends communication signals to and/or receives communication signals from one or more other devices. Devices are “communicatively connected” if the devices are able to send and/or receive data via a communication link. “Electronic communication” refers to the transmission of data via one or more signals between two or more electronic devices, whether through a wired or wireless network, and whether directly or indirectly via one or more intermediary devices.

The features and functions described above, as well as alternatives, may be combined into many other different systems or applications. Various alternatives, modifications, variations or improvements may be made by those skilled in the art, each of which is also intended to be encompassed by the disclosed embodiments.

1. A system for enforcing occupancy for a facility, the system comprising:
a plurality of client electronic devices, each associated with one or more individuals who have access to a facility;
an access monitoring system;
a computing device in communication with at least a portion of the plurality of the client electronic devices and the access monitoring system; and
a computer-readable storage medium comprising one or more programming instructions that, when executed, cause the computing device to:
determine a health risk rating for a facility,
determine a maximum occupancy for the facility,
determine a risk-adjusted occupancy for the facility by adjusting the maximum occupancy based on the health risk rating,
determine an estimated occupant number for the facility during a time period using the access monitoring system,
determine, based on the risk-adjusted occupancy and the estimated occupant number, whether one or more individuals need to exit the facility,
in response to determining that one or more individuals need to the exit the facility, identify the one or more individuals from a plurality of individuals who are present at the facility at the time period, and
notify the one or more individuals who are identified to exit the facility.

2. The system of claim 1, wherein the access monitoring system comprises an indoor location tracking system comprising one or more wireless access points in communication with one or more of the plurality of client electronic devices.

3. The system of claim 1, wherein the access monitoring system comprises a geofence system in communication with at least a portion of the plurality of client electronic devices, wherein the geofence system comprises one or more receivers configured to receive location-based data from one or more of the plurality of client electronic devices.

4. The system of claim 1, wherein the access monitoring system comprises a physical access control system comprising one or more reader devices that are configured to receive one or more access credentials from one or more individuals seeking access to the facility.

5. The system of claim 1, wherein the one or more programming instructions that, when executed, cause the computing device to determine a health risk rating for a facility comprise one or more programming instructions that, when executed, cause the computing device to:
access information from one or more health metrics data stores associated with the facility; and
apply a framework to at least a portion of the information that is accessed.

6. The system of claim 5, wherein the information from the one or more health metrics data stores comprises:
a trend value associated with a health condition over a period of time and for a geographic location;
a measure of a number of cases of the health condition over the period of time and for the geographic location; and
a positivity rate associated with the health condition over the period of time and for the geographic location.

7. The system od claim 1, wherein the one or more programming instructions that, when executed, cause the computing device to determine a maximum occupancy for the facility comprise one or more programming instructions that, when executed, cause the computing device to identify the maximum occupancy for the facility from one or more capacity data stores associated with the facility.

8. The system of claim 1, wherein the one or more programming instructions that, when executed, cause the computing device to determine a risk-adjusted occupancy for the facility comprise one or more programming instructions that, when executed, cause the computing device to:
determine whether the health risk rating for the facility is at a highest level;
in response to the health risk rating for the facility being at the highest level, identify an essential personnel capacity for the facility, wherein the essential personnel capacity represents a number of essential works for the facility; and
assign the essential personnel capacity to the risk-adjusted occupancy.

9. The system of claim 1, wherein the one or more programming instructions that, when executed, cause the computing device to determine a risk-adjusted occupancy for the facility comprise one or more programming instructions that, when executed, cause the computing device to:
determine whether the health risk rating for the facility is at a lowest level;
in response to the health risk rating for the facility being at the lowest level, identify a normal capacity for the facility;
assign the normal capacity to the risk-adjusted occupancy.

10. The system of claim 1, wherein the one or more programming instructions that, when executed, cause the computing device to determine a risk-adjusted occupancy for the facility comprise one or more programming instructions that, when executed, cause the computing device to:
determine whether the health risk rating for the facility is between a lowest level and a highest level;
in response to the health risk rating for the facility being between a lowest level and a highest level:
identify a normal capacity for the facility, and
assign a certain percentage of the normal capacity to the risk-adjusted occupancy.

11. The system of claim 1, wherein the one or more programming instructions that, when executed, cause the computing device to determine an estimated occupant number for the facility at the time period comprise one or more programming instructions that, when executed, cause the computing device to perform one or more of the following:
identify a number of individuals who have submitted access requests to access the facility during the time period;
receive, from an indoor location tracking system, an estimate of a number of electronic devices in communication with one or more wireless access points at the facility during the time period;
receive, from a geofence system, an estimate of a number of electronic devices within a geofence during that time period, wherein the geofence encompasses the facility; or
receive an estimate of a number of individuals who have presented one or more access credentials to one or more reader devices at the facility over the time period.

12. The system of claim 1, wherein the one or more programming instructions that, when executed, cause the computing device to determine an estimated occupant number for the facility at a time period comprise one or more programming instructions that, when executed, cause the computing device to:
identify a first estimate as a number of individuals who have submitted access requests to access the facility during the time period;
receive, from an indoor location tracking system, a second estimate comprising an estimate of a number of electronic devices in communication with one or more wireless access points at the facility during the time period;
receive, from a geofence system, a third estimate comprising a number of electronic devices within a geofence during that time period, wherein the geofence encompasses the facility;
receive a fourth estimate of a number of individuals who have presented one or more access credentials to one or more reader devices at the facility over the time period; and
average the first estimate, the second estimate, the third estimate, and the fourth estimate.

13. The system of claim 1, wherein the one or more programming instructions that, when executed, cause the computing device to determine, based on the risk-adjusted occupancy and the estimated occupant number, whether one or more individuals need to exit the facility comprise one or more programming instructions that, when executed, cause the computing device to determine whether the risk-adjusted occupancy is greater than the estimated occupant number.

14. The system of claim 1, wherein the one or more programming instructions that, when executed, cause the computing device to identifying the one or more individuals from a plurality of individuals who are present at the facility at the time period comprise one or more programming instructions that, when executed, cause the computing device to identify the one or more individuals as those associated with a priority designation for the facility that indicates they are not essential personnel.

15. The system of claim 1, wherein the one or more programming instructions that, when executed, cause the computing device to notify the one or more individuals who are identified to exit the facility comprise one or more programming instructions that, when executed, cause the computing device to perform one or more of the following:
cause an email to be sent to an email address of each of the one or more individuals who are identified;
cause a text message to be sent to an electronic device of each of the one or more individuals who are identified, or
cause a push notification to be displayed on an electronic device of each of the one or more individuals who are identified.

16. A method of enforcing occupancy for a facility, the method comprising:
by an electronic device:
determining a health risk rating for a facility,
determining a maximum occupancy for the facility,
determining a risk-adjusted occupancy for the facility by adjusting the maximum occupancy based on the health risk rating,
determining an estimated occupant number for the facility during a time period using the access monitoring system,
determining, based on the risk-adjusted occupancy and the estimated occupant number, whether one or more individuals need to exit the facility,
in response to determining that one or more individuals need to the exit the facility, identifying the one or more individuals from a plurality of individuals who are present at the facility at the time period, and
notifying the one or more individuals who are identified to exit the facility.

17. The method of claim 16, wherein determining a health risk rating for a facility comprises:
accessing information from one or more health metrics data stores associated with the facility; and
applying a framework to at least a portion of the information that is accessed,
wherein the information from the one or more health metrics data stores comprises:
a trend value associated with a health condition over a period of time and for a geographic location,
a measure of a number of cases of the health condition over the period of time and for the geographic location, and
a positivity rate associated with the health condition over the period of time and for the geographic location.

18. The method of claim 16, wherein determining a risk-adjusted occupancy for the facility comprises:
determining whether the health risk rating for the facility is at a highest level;
in response to the health risk rating for the facility being at the highest level, identifying an essential personnel capacity for the facility, wherein the essential personnel capacity represents a number of essential works for the facility;
assigning the essential personnel capacity to the risk-adjusted occupancy.

19. The method of claim 16, wherein determining a risk-adjusted occupancy for the facility comprises:
determining whether the health risk rating for the facility is at a lowest level;
in response to the health risk rating for the facility being at the lowest level, identifying a normal capacity for the facility;
assigning the normal capacity to the risk-adjusted occupancy.

20. The method of claim 16, wherein determining a risk-adjusted occupancy for the facility comprises:
determining whether the health risk rating for the facility is between a lowest level and a highest level;
in response to the health risk rating for the facility being between a lowest level and a highest level:
identifying a normal capacity for the facility, and
assigning a certain percentage of the normal capacity to the risk-adjusted occupancy.

21. The method of claim 16, wherein determining an estimated occupant number for the facility at a time period comprises one or more of the following:
identifying a number of individuals who have submitted access requests to access the facility during the time period;
receiving, from an indoor location tracking system, an estimate of a number of electronic devices in communication with one or more wireless access points at the facility during the time period;
receiving, from a geofence system, an estimate of a number of electronic devices within a geofence during that time period, wherein the geofence encompasses the facility; or
receiving an estimate of a number of individuals who have presented one or more access credentials to one or more reader devices at the facility over the time period.

22. The method of claim 16, wherein determining an estimated occupant number for the facility at a time period comprises:
identifying a first estimate as a number of individuals who have submitted access requests to access the facility during the time period;
receiving, from an indoor location tracking system, a second estimate comprising an estimate of a number of electronic devices in communication with one or more wireless access points at the facility during the time period;
receiving, from a geofence system, a third estimate comprising a number of electronic devices within a geofence during that time period, wherein the geofence encompasses the facility;
receiving a fourth estimate of a number of individuals who have presented one or more access credentials to one or more reader devices at the facility over the time period; and
averaging the first estimate, the second estimate, the third estimate, and the fourth estimate.

23. The method of claim 16, wherein determining, based on the risk-adjusted occupancy and the estimated occupant number, whether one or more individuals need to exit the facility comprises determining whether the risk-adjusted occupancy is greater than the estimated occupant number.

24. The method of claim 16, wherein identifying the one or more individuals from a plurality of individuals who are present at the facility at the time period comprises identifying the one or more individuals as those associated with a priority designation for the facility that indicates they are not essential personnel.

25. The method of claim 16, wherein notifying the one or more individuals who are identified to exit the facility comprises one or more of the following:
causing an email to be sent to an email address of each of the one or more individuals who are identified;
causing a text message to be sent to an electronic device of each of the one or more individuals who are identified, or
causing a push notification to be displayed on an electronic device of each of the one or more individuals who are identified.
